(19) United States
(12) Patent Application Publication
(10) Pub. No.: US 2006/0260939 A1
(43) Pub. Date: Nov. 23, 2006

(54) Detecting apparatus

(76) Inventors: Ian Anderson, Cambridge (GB); Claude Paul Kaplan, Royston (GB); Toby Trevor Fury Mottram, Cambridge (GB); Stephen Russell Taylor, Cambridge (GB); Maria Nieves Velasco Garcia, Bedford (GB)

Correspondence Address: DYKEMA GOSSETT PLLC, FRANKLIN SQUARE, THIRD FLOOR WEST, 1300 I STREET, NW, WASHINGTON, DC, 20005, US

(21) Appl. No.: 10/529,771

(22) PCT Filed: Oct. 9, 2003
(86) PCT No.: PCT/GB03/04391

§ 371 Date: May 2, 2006

(30) Foreign Application Priority Data
Oct. 9, 2002 (GB) 0223476.3

(51) Int. Cl. G01N 33/487

(52) U.S. Cl. 204/403.01

(57) ABSTRACT

An automated detection apparatus for testing biological samples from a plurality of individual animals. The apparatus comprises a detecting unit having a sample inlet and a sample outlet, and a biosensitive sensor comprising a biosensitive medium for indicating the concentration of at least one biological compound within each biological sample. The biosensitive medium is provided with at least one active biosensor region. Means expose each region, when in use, to a biological sample thereby detecting the concentration of the biological compound.

This invention relates to an apparatus for detecting biological agents in animal body fluids, for instance milk.

In recent times it has been realised that there is a benefit in monitoring collected animal body fluids, such as milk from dairy cattle, for the presence of certain chemicals or hormones. For example, it can be beneficial to monitor the milk of a cow to detect the presence and level of progesterone in order to determine its ovulating cycle. Alternatively, there may be a need to monitor the milk to detect for other types of bio-markers, such as chemical imbalances that are indicative of a disease in the cow or other animal.

Monitoring collected animal body fluids for the presence and concentration of hormones, and in particular progesterone, is known. By frequent analysis of progesterone levels in milk samples from a particular animal, ovulation cycles can be mapped. Similarly monitoring of luteinising hormone levels in milk samples gives another method for mapping an animal's ovulation cycle.

Examples of other types of bio-markers that are known to benefit from monitoring, include: NAGase activity, which can indicate that an animal has an inflammatory response for example sub-clinical mastitis; and ketone levels, which indicates whether an animal may have ketosis. Bio-markers can also indicate response to the presence of a disease, for example bovine viral diarrhoea virus (BVDV) or Leptosporosis (Weil's disease). Detection of disease vectors can lead to early treatment of the disease and the prevention of spread of the disease to other animals.

In one known progesterone monitoring method, a solid base immunoassay is adopted. This technique makes use of a biosensor. The biosensor comprises a solid supporting medium provided with test regions to which a known quantity of a specific antibody (monoclonal antibody) is attached. A mixture of the milk sample and a second labelled progesterone solution is added to the biosensor. Both labelled and unlabelled progesterone will bind to the antibody regions in competition with each other.

The labelled progesterone is added at a known fixed concentration. The labelling in this case allowing electrochemical measurement.

At least one control sample having a known concentration of progesterone may be provided as part of each assay for calibration purposes. If necessary a plurality of control samples at a range of expected concentrations of (unlabelled) progesterone may be used to construct a “standard” concentration curve from which the concentration in the sample is interpolated.

The biosensor is washed to remove the portion of the sample mixture not bound to the biosensor. The washed biosensor is then exposed to a substrate and allowed to incubate for a predetermined time. The incubated biosensor is maintained in a detection unit where the substrate is added and by products of the substrate are measured electrochemically.

Sampling of farm animals and measurement of these bio-markers by specialists (in particular veterinarians) on the farm, within veterinary practices and within analytical laboratories is typically expensive and time-consuming.

Specialist knowledge and expertise is needed: to identify from which animals to sample and measure the bio-marker; to take a sample of body fluid; to undertake the assay to measure the bio-marker; and to process the results of the assay and suggest appropriate action.

The farm environment presents additional problems. Testing on farm premises has been seen as unfeasible due to the difficulty of achieving satisfactory precision and because of the time the farmer can afford to spend performing the tests manually.

Even manual monitoring of milk has to date proved impractical. There has been a considerable amount of research and development in terms of automating animal milk collection. It has hitherto proved impossible to gather milk automatically and then to monitor for the presence of bio-markers (biological or chemical agents) in any automatic manner. Typically the manual monitoring and processing is extremely time consuming and inconvenient.

Taking and then labelling samples and finally performing the appropriate assay within the farm environment still requires a good deal of specialist knowledge, time and effort. As a consequence, although many bio-markers are known, the financial and time costs involved in measuring these bio-markers means that these bio-markers are rarely routinely measured within farm animals.

The present invention seeks to provide an apparatus that overcomes or at least ameliorates some of the current problems associated with the monitoring of milk collected from animals such as dairy cattle.

In accordance with the present invention there is provided an automated detection apparatus for testing biological samples from a plurality of individual animals, the apparatus comprising: a detection unit having a sample inlet and a sample outlet, and a biosensitive sensor comprising a biosensitive medium for indicating the concentration of at least one biological compound within each biological sample, wherein the biosensitive medium is provided with at least one active biosensor region; and means for exposing each region, when in use, to a biological sample thereby detecting the concentration of said biological compound.

By automating the exposure of successive active biosensor regions to the corresponding samples, much of the inconvenience of conventional monitoring systems can be avoided.

The present invention will now be described with reference to the accompanying drawings, in which:

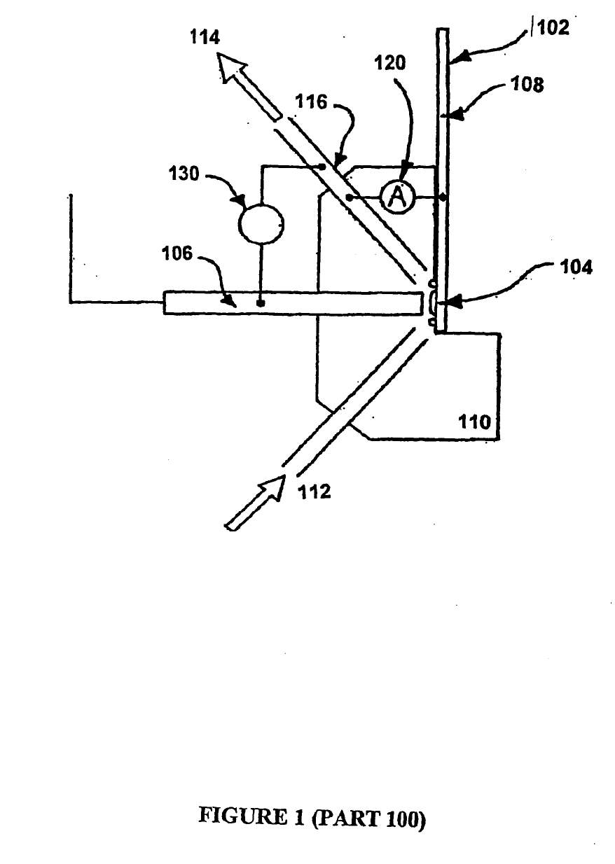

FIG. 1 shows a detection unit according to a first embodiment of the present invention;

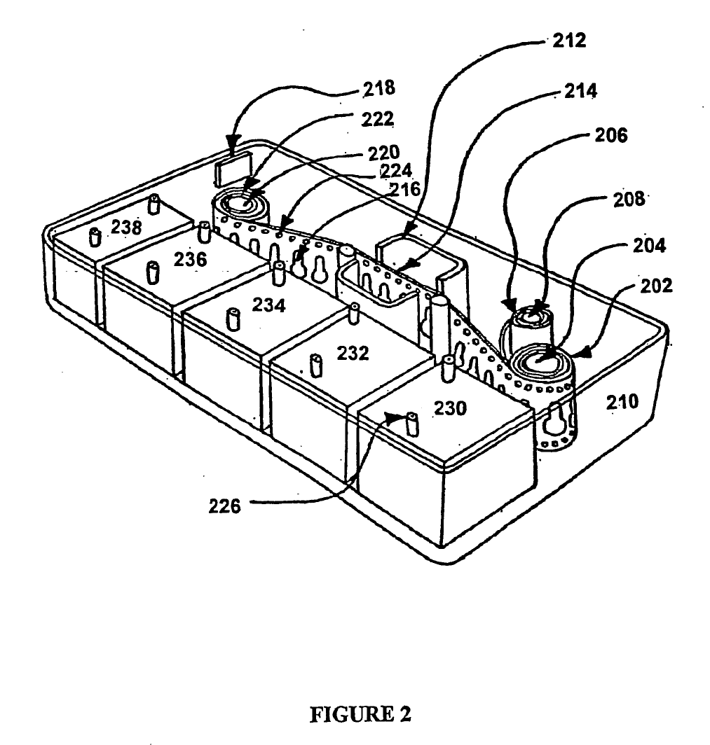

FIG. 2 illustrates a cassette arrangement according to a second embodiment of the present invention;

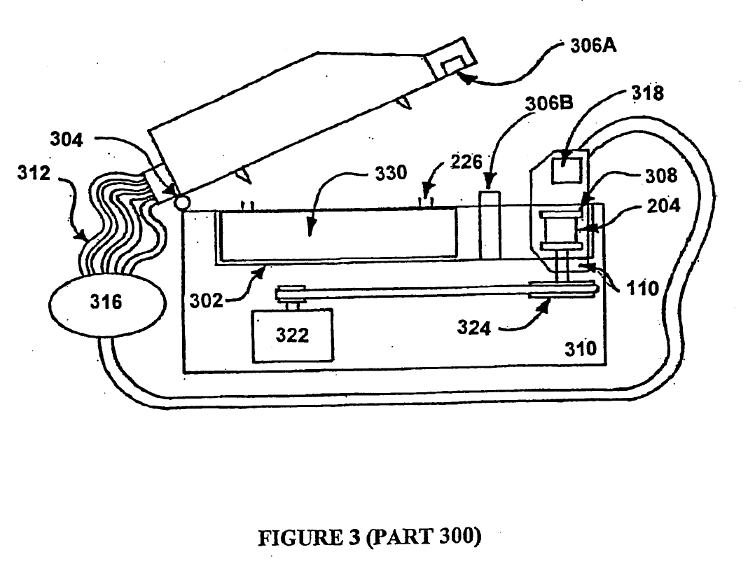

FIG. 3 shows a side view of the cassette of FIG. 2 in place in a milking apparatus; and

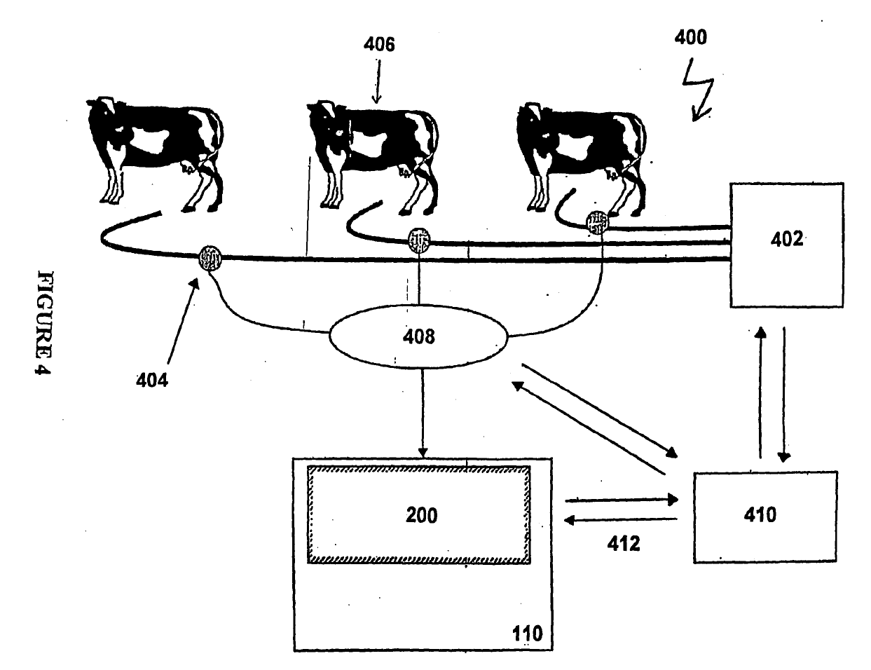

FIG. 4 illustrates an implementation of the sensor cassette according to the present invention.

Although a device in accordance with the present invention may be adapted to measure bio-markers from a number of different farm animals within a number of different body fluids, the invention will be described by way of illustration with respect to measuring bio-markers within the milk of dairy cows during or after milking within a milking parlour.

In a first embodiment 100, the biosensor 104 is provided on a card 102. The card 102 is conveyed into a detection unit 110. The detection unit 110 is provided with a reference electrode 106, a sample inlet port 112 and a sample outlet port 114. The card 102 is provided with electrical contacts 108 so that when in place in the detection unit 110, the card 102 acts as a working electrode. In this example a single sensor is on each card, but the system can be arranged to have more than one sensor on each card.

FIG. 1 illustrates the detection unit 110 of the first embodiment. Here inlet 112 and outlet ports 114 are pipes, preferably and made of a stainless steel or similar conductor and plastic inlet. The reference electrode 106 is made of silver/silver chloride (Ag/AgCl). This electrode 106 may be part of a disposable sensor. A potential difference is applied between the auxiliary electrode 116 and reference electrode 106 and the current is measured in the circuit formed by the auxiliary electrode 116, a power source 130 and the biosensor card 102 (ie. working electrode 108). In FIG. 1, the sample outlet port 114 has been configured as an auxiliary electrode 116 and coupled to the working electrode 108. The current is measured by an ammeter 120 or similar device between the working 108 and auxiliary electrodes 116. The arrangement is shown in a vertical orientation, but could be adapted to a horizontal orientation with samples dropped under gravity onto the sensor. This arrangement allows for free surface mixing which may have benefits in some cases.

The card 102 may be conveyed in any conventional manner, for instance a card mounting arrangement as might be seen in a slide projector or can be one of a strip of rigid cards. Once a sample has been tested, the card is removed from the detection unit 110: in one version of this system, the card simply drops under gravity once tested.

The detection unit 110 is suitable for installation with an automated milking apparatus. Samples from the milk collected from a given animal are ordered and stored. These samples may be tested substantially in real time or ‘offline’ at sometime after the milking procedure. The biosensor card system of the first embodiment can be automatically ordered to facilitate testing. Details of measurements corresponding to each given animal are transmitted to an animal database. In a fully automated system, the test measurements are transmitted electronically as data signals for storage in a computer database.

Rather than single or multiple use biosensor cards, it may be preferable to provide the biosensor as a frame on a biosensor film. In a second embodiment of the present invention, biosensor film is provided in a cassette housing. Biosensor film can be supplied in much the same way as photographic film. It may be a flexible film, or may be formed by rigid sensor cards attached to one another in a flexible manner.

In common with photographic film, the biosensor film is sufficiently flexible to allow winding about a spool or is packed so the sensor bases are not deformed. The biosensor film may be protected by a removable foil coating. Although not shown, the film may be supplied in a canister suitable for insertion in a film receiving socket. The biosensor film may be reused, typically after appropriate reconditioning.

FIG. 2 illustrates the cassette embodiment 200. Fresh biosensor film 202 is wound around a feed spool 204. The foil coating 206 is peeled off and wound about a further spool 208. The cassette 210 provides a recess 212 whereby a predetermined length (a frame) of film 214 is exposed. Each frame 214 exposes a plurality of active biosensor regions (or slots) 216. The exposed frame 214 is exposed to a milk sample from a given animal. Each cassette 210 may be tagged with a unique identifier 218, for example a radiofrequency ID tag (RFID). Once the film 202 has been sufficiently exposed to the sample, the cassette arrangement then advances the film to expose a fresh frame 214. A drive spool 220 is provided and exposed film 222 is wound onto the drive spool 220.

The film may be provided with sprocket perforations 224, which engage a sprocket wheel (not shown). The sprocket wheel allows the film 202 to be moved accurately and incrementally between successive frames 214. A position sensor may be employed to confirm that this is the case.

The cassette in FIG. 2 is further provided with a plurality of reservoirs 230,232,234,236,238 that contain all or at least some of the reagents required to implement a testing technique. Examples of appropriate testing techniques include chemical, biochemical and immuno-assay. As an alternative reagents may also be incorporated into the biosensor measurement device itself.

In the case illustrated in FIG. 2, the cassette 210 includes a labelling reagent reservoir 232, a control sample reservoir 236, a wash reservoir 230, a substrate reservoir 234 and a buffer reservoir 238: all reagents which may be used in the competitive immunoassay technique described above. Preferably the cassette 210 is provided with sufficient reagent and film to effect a considerable number of measurements before it is exhausted; a typical number being around 100 measurements.

The reagents and the biosensors used typically have an operative range of temperatures. The cassette may further be provided with a temperature control mechanism (not shown) for maintaining the temperature of the cassette at a specified level or within a predetermined range.

The cassette of FIG. 2 is disposed within a cassette receiving recess 302 of a milking apparatus 300. A side view of the cassette 210 in place in such a receiving recess 302 is shown in FIG. 3. The cassette 210 can therefore be inserted and removed from the biosensor measurement device 110.

Each reagent reservoir 330 is sealed. To allow access to the reagent contained within the reservoir 330 may conveniently be provided with a septum 226. Once a cassette has been introduced in the recess 302 of the milking apparatus, a cover portion 320 is fixed in place over the cassette 210. In FIG. 3, the cover 320 is attached to the body 310 of the milking apparatus 300 by a hinge 304 and held in place by a clamping mechanism 306A,306B. In other implementations, the cover may be fully removable or slidably movable. Once the cover 320 is in place over the cassette, a fluid connection is established to each reservoir.

When in the biosensor measurement device 110, the sensor regions 216 can be moved into and out of a reaction chamber 308 as required, thereby allowing a specific bio-marker to be measured in a number of milk samples.

The cassette may be arranged so that it may easily be reused. In particular, the biosensors themselves and/or the biosensor film may be reusable. It may also be arranged so that it can be placed in different orientations. For example it may be arranged so that, in use, samples can be dropped onto appropriately exposed sensors, using gravity and a dropping process to avoid the need for an enclosed chamber.

In the illustrated embodiment, the cover is provided with a plurality of channels 312. At one end of each channel 312 is provided a piercing tip 314, which pierces the septum 226 sealing a corresponding reservoir 330. The remaining end of the channel 312 is connected via a pump 316 to the reaction chamber 308. Each channel 312 therefore establishes fluid contact via the cover 320 to the reaction chamber 308. The pump 316 allows selective passage of reagents into the reaction chamber 308.

As described in relation to FIG. 2 the biosensor film 202 is guided through the biosensor measurement device 110 between two spools 204,220. The spools themselves are driven by a motor 322. The motor 322 provides rotational drive to the drive spool 220 by means of a capstan 324.

The milking apparatus may also be provided with a tag interface (not shown). The interface would allow data stored on the identification tag to be read, edited or even erased when the cassette is to be reused. Data stored on the tag may usefully include specific information about the cassette, for example: a batch code; the nature and number of sensors provided on the film; calibration data; the data relating to the manufacture of the cassette; and/or reel usage information (such as frame number).

The interface allows the cassette to communicate such information to the bio-sensor measurement device.

FIG. 4 illustrates how use of the sensor cassette embodiment 200 is implemented within milking arrangement 400. The conventional milking apparatus 402 is provided with sampling devices 404, which take samples from the milk produced by each cow 406. The samples are ordered and stored for later retrieval. The samples are tested as previously described using, for example, a biosensor cassette 200.

The automated sample processing arrangement 400 includes: a herd management processing system 410; a plurality of sampling devices 404; a sample storage device 408; at least one bio-sensor measurement device 110; a sensor medium 202; and an animal identification device (not shown).

The processing system, for example a computer or a microprocessor device, has a memory unit, the memory unit storing: a database of information on individual animals; a plurality of mathematical models of bio-marker properties; and interface software, for interfacing with the sample storage device and the plurality of bio-sensor measurement devices. Current implementations of the processing system include: embedded PCs; PC104 expansion cards; and RCom. The operating system used may be any convenient OS, for example DOS, MS Windows, UNIX/Linux, Apple, Symbian EPOC or PalmOS. The data base may access remotely located data.

The processing system is programmed to receive and update information 412 held on the animal database. Examples of the information held on the database include: age, calving information, and previous bio-marker measurements. The processing system is also programmed to use the mathematical models to relate the measured concentration of specified bio-markers to fertility, wellness or disease status.

Sampling devices in a milking apparatus are attached either to each milk line or to another device within the milk line. In operation, the sampling devices divert a sample of milk onto a sample line. The diversion of samples may be effected during or after milking. The sampling devices may take a milk sample from every cow being milked or only designated cows: it may take samples continually or at a specified time point and it may be attached to a conventional or robotic milking system.

The sample storage device is a device for temporarily storing one or more milk sample. This device is able to receive milk samples from the one or more sampling device, to store the samples and, when instructed by the herd management processing system, to direct the sample to the bio-sensor measurement devices. The sample may also be directed to another device (for example, a collection device) or for waste disposal.

The bio-sensor measurement devices are able to take a sample of milk directly from the sampling device (or from the sample storage device) and to conduct a chemical, biochemical or physical assay to measure one or more bio-marker. For this purpose each bio-sensor measurement device includes a reaction chamber, where the assay may be carried out. The data output from the assay is communicated to the herd management processing system. In the case of measuring for a number of bio-markers, the assay will require the use of a sensor medium, which can be inserted into the bio-sensor measurement device and interchanged in accordance with the bio-marker being assayed.

The sensor medium is conveniently disposable or reusable. The sensor medium comprises a plurality of biosensitive regions, which when inserted into the bio-sensor measurement device operate as one or more biosensors.

Several types of bio-sensor measurement device will accommodate a sensor media with biosensitive regions, thereby enabling the measurement of a range of bio-markers of interest. The sensor medium typically contains: a substrate (for example PVC) upon which the biosensitive regions are disposed. Depending upon the particular assay (or assays) to be performed, each biosensitive region (or biosensor) may include one or more key elements required to measure one or more bio-marker, for instance assay solutions, electrodes (often made of carbon) or gold, or fixed antibodies.

As described above the biosensitive medium may be provided within a cassette or on a card.

The incorporation of antibodies (either deposited on the bio-sensitive region or in free solution) allows a specific immunoassay to take place within the reaction chamber and for a specific bio-marker to be measured. In certain cases, the bio-sensor measurement device includes a control reaction chamber in which measurements from a bio-sensor (in the absence of one component of the assay) will be used to remove a substantial proportion of any background signal from the milk.

Clearly, different bio-sensors incorporating different antibodies can be utilised within the same reaction chamber (within the same bio-sensor measurement device) to measure different molecules. For example a single bio-sensor measurement device may both measure progesterone using a progesterone sensor and also measure BVDV using a BVDV-sensor.

Some assays do not require a bio-sensor (or use a bio-sensor but require no antibodies) and will measure a bio-marker directly in the milk utilising a chemical or physical reaction. Examples of such assays include: an enzymatic reaction catalysed by an enzyme in the milk; surface plasma resonance or a specific wavelength of the electromagnetic spectrum correlating to the concentration of a known bio-marker.

Preferably, the automated sample processing arrangement includes a cow identification device, which allows the identification of which cow is being milked and (if required) which stall the cow is being milked in. Identification data may be gathered automatically, for example the animal may be fitted with a transponder whose signal is received by an antenna coupled to the identification device. Alternatively the data may be gathered manually, for instance through data entry into a mobile terminal device with a communication link to the herd management processing device or through a conventional computer keyboard plugged into the herd management processing system. The identification device will therefore communicate directly or indirectly with the herd management system.

The operation of an automated milk monitoring device in accordance with a further aspect of the present invention will now be described.

The cow identification device gathers cow identification information (whether manually or automatically) thereby recognising which cow is being milked and, if appropriate, in which milking stall.

Cow identification information is transferred to the herd management processing system, which accesses the cow database to retrieve data relating to the identified cow and the mathematical models for specified bio-marker properties. The processing system then analyses: information on the cow; parameters set by the farmer, the models of specified bio-markers; measurement regimes and other information. Next the processing system determines whether a sample of milk from that cow should be used for measuring one or more bio-marker

The sampling device takes a sample of milk from the milk line while the, now identified, cow is being milked. This sampling may occur for all cows or for only specified cows. As described above, the sample from one or more sampling devices may be directed to the sample storage device.

Depending upon the instructions received from the herd management processing system, the sample storage device instigates one of a number of actions. The actions including: disposal of the milk sample; temporary storage of the sample, for instance when for later direction to a bio-sensor or other measurement device; directing the sample, or a part of the sample, to one or more bio-sensor or other type of measurement devices; and directing the sample to a sample collection (or other permanent storage) apparatus.

Alternatively the sample may be directed straight to the bio-sensor measurement device thereby bypassing the storage device altogether.

When samples are directed to the bio-sensor measurement device, the measurement device conducts a chemical, biochemical or physical assay and measures at least one specific bio-marker in that milk sample. The herd management processing system determines which bio-sensor measurement device and which bio-markers are to be measured. The data output of the assay will be communicated to the herd management processing system

The herd management processing system will then process the results of the assay, using the embedded mathematical models of specified bio-markers and stored animal data relating to that specific cow. The processing system is preferably programmed to present a graphical user interface to allow the farmer to access the acquired information and ultimately to assess the status of his herd. If any urgent actions are required, the processing system is advantageously programmed to alert the operator and to suggest what action may be required, for example; “cow A3 (currently in stall 5) is ovulating, contact the AI (artificial insemination) professional within 24 hours”, or “cow F5 is not ovulating as normal, contact the veterinarian”.

The processing system may furthermore be in communication with wireless and/or wire networks of computing devices. The processing system can then generate and send text messages directly to a wireless communicator device (for instance, a mobile telephone or a personal communication device) to report the status of an individual cow or of the whole herd. Likewise processing system can send a request for action directly to a third party (for example an email message to a veterinarian or an AI professional).

By providing an integrated wash system, which is coordinated with conventional milking machine wash cycles, the sampling devices, the sample storage device and bio-sensor measurement devices can be washed out between milk sampling and/or at the completion of the milking of the herd.

As might be expected, the processing system is preferably programmed to be able to change the sensitivity and frequency of measurements of any given bio-marker. The software running on the processing system is preferably capable of learning and adapting to the requirements of each individual cow.

1. An automated detection for testing biological samples from a plurality of individuals animals, the apparatus comprising:
a detection unit having a sample inlet and a sample outlet, and a biosensitive sensor comprising a biosensitive medium for indicating the concentration of at least one biological compound within each biological sample, wherein the biosensitive medium is provided with at least one active biosensor region; and
means for exposing each region, wherein in use, to a biological sample thereby detecting the concentration of said biological compound.

2. The apparatus as claimed in claim 1, wherein the biosensitive medium indicates the concentration of said at least one biological compound in accordance with a solid based immunoassay technique.

3. The apparatus as claimed in claim 1, wherein the biosensitive medium indicates the concentration of progesterone.

4. The apparatus as claimed in claim 1, wherein the detection unit includes a plurality of electrodes, which introduce an electrical potential across at least a portion of the sample under test and wherein the concentration of said at least one biological compound is detected electrochemically.

5. The apparatus as claimed in claim 4, wherein the plurality of electrodes includes a working electrode and the sensor is electrically coupled to a working electrode.

6. The apparatus as claimed in claim 4, wherein the plurality of electrodes further includes a reference electrode and wherein the concentration of said at least one biological compound is detected by measuring the current flowing in a circuit including the reference electrode, the sample under test and the first electrode.

7. The apparatus as claimed in claim 6, wherein the inlet and outlet ports are formed of a conductive material and wherein the outlet port is electrically coupled to the reference electrode as an auxiliary electrode.

8. The apparatus as claimed in claim 1, wherein the sensor is a biosensor card and wherein the means for exposing each region is a card mounting arrangement.

9. The apparatus as claimed in claim 2, wherein the sample inlet and outlet ports are pipes.

10. The apparatus as claimed in claim 1, wherein the sensor is a biosensor film and wherein the means for exposing each region comprises a film mounting arrangement.

11. The apparatus as claimed in claim 1, wherein the biosensor film is provided with sprocket perforations for engaging a sprocket wheel, which in use drives the film.

12. The apparatus as claimed in claim 3, wherein the means for exposing each region further comprises: a plurality of reagent reservoirs, each containing a testing reagent, and a plurality of fluid channels for conveying said reagents into said detection unit.

13. The apparatus as claimed in claim 3, wherein the means for exposing each region is disposed in a cassette housing.

14. The apparatus of as claimed in claim 3, further comprising means for controlling the handling of the sample to ensure disposal or storage after testing.

15. A biosensor cassette for testing biological samples from a plurality of individual animals, the cassette comprising:
a housing; and
a biosensor film;
wherein the film is provided within the housing, the film in use being capable of selective exposure to biological samples in a detection chamber thereby facilitating the detection of concentrations of a biological compound within said samples.

16. The cassette as claimed in claim 15, wherein the cassette further comprises at least one reagent reservoir for storing reagents, wherein said at least one reservoir is disposed within the housing and wherein the reagents are supplied to the detection chamber.

17. The cassette as claimed in claim 14, wherein the biosensor film is provided with sprocket perforations for engaging a sprocket wheel, which in use drives the film.

18. The cassette as claimed in claim 15, further comprising an identifier tag for storing identification data.

19. The cassette as claimed in claim 18, wherein data stored on the tag includes a batch code, the date of manufacture and reel usage information.

20. A milking apparatus incorporating a detection apparatus as claimed claim 1, wherein the biological samples are milk samples and wherein the automatic apparatus comprises milking device, a detection apparatus recess for receiving said detection apparatus and a testing controller for controlling the testing of milk samples.

21. The milking apparatus as claimed in claim 19, wherein the detection apparatus is a biosensor cassette and wherein the recess receives the cassette, the automatic milking apparatus further comprising a drive mechanism, wherein the cassette recess is provided with at least one capstan for engaging with a spool provided in the cassette and for controllably driving the biosensor film past the milk sample, wherein the drive mechanism drives said at least one capstan, and thereby advances the film, under the control of the testing controller.

22. The milking apparatus as claimed in claim 20, further comprising a tag interface for allowing data stored on the identification tag to be read and edited.

23. (canceled)
